(19) United States
(12) Patent Application Publication
(10) Pub. No.: US 2025/0345568 A1
(43) Pub. Date: Nov. 13, 2025

(54) REPOSITIONING SHEATH

(71) Applicant: BOSTON SCIENTIFIC SCIMED, INC., Maple Grove, MN (US)

(72) Inventors: Judith Tiferes Wang, Shoreview, MN (US); Qian Liu, Plymouth, MN (US); Brian R. Reynolds, Ramsey, MN (US)

(73) Assignee: BOSTON SCIENTIFIC SCIMED, INC., Maple Grove, MN (US)

(21) Appl. No.: 19/205,301

(22) Filed: May 12, 2025

Related U.S. Application Data
(60) Provisional application No. 63/646,146, filed on May 13, 2024.

(51) Int. Cl. A61M 25/06; A61M 25/00

(52) U.S. Cl. CPC A61M 25/0662; A61M 25/0045; A61M 25/0053; A61M 25/0097; A61M 2210/12

(57) ABSTRACT

A mechanical circulatory support system may include a blood pump, an elongate shaft, a first sheath, and a second sheath. The elongate shaft may be coupled with the blood pump and extend proximally from the blood pump. The first sheath may extend over the elongate shaft and include a lumen with an inner diameter. The second sheath may extend over the elongate shaft and include an outer diameter that is less than the inner diameter of the lumen of the first sheath. The first sheath and the second sheath are configured to longitudinally adjust over the elongate shaft and one or both of the first sheath and the second sheath may include a flexible portion configured to extend through a vascular access site when a more rigid distal portion is in of the sheath is in a blood vessel.

### CROSS REFERENCE TO RELATED APPLICATIONS

The present application claims the benefit of U.S. Provisional Patent Application Ser. No. 63/646,146, filed May 13, 2024, the disclosure of which is incorporated herein by reference.

### TECHNICAL FIELD

The present disclosure pertains to a sheath configuration for a mechanical circulatory support system. More particularly, the present disclosure pertains to a sheath of a mechanical circulatory support system for use at a vascular access site.

### BACKGROUND

In various procedures for delivering intravascular medical devices, an introducer sheath is inserted into a blood vessel of a patient, for example a femoral artery, and one or more medical devices may be advanced through the sheath and into the patient's vasculature. In various instances, the medical devices include catheters or other devices, such as a blood pump. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing and using medical devices and systems.

### BRIEF SUMMARY

This disclosure provides design, material, manufacturing method, and use alternatives for medical devices, including percutaneous blood pumps and associated devices.

In a first example a sheath may include an elongate tube, the elongate tube having a proximal end, a distal end, a lumen extending between the proximal end and the distal end, and an outer surface defining an outer diameter, and wherein the outer diameter may be configured to pass through an introducer lumen of an introducer sheath and the elongate tube has a first portion with a first stiffness and a second portion located distal of the first portion with a second stiffness that is greater than the first stiffness.

Alternatively or additionally to any of the examples above, the elongate tube may have a third portion located proximal of the first portion with a third stiffness that is greater than the first stiffness.

Alternatively or additionally to any of the examples above, the first portion may be configured to extend through an opening in a skin of a subject when the second portion extends into a blood vessel of the subject.

Alternatively or additionally to any of the examples above, the first stiffness of the first portion of the elongate tube may be configured to adapt to a contour of skin of the subject at the opening in the skin.

Alternatively or additionally to any of the examples above, the first portion of the elongate tube may be made from a material having a durometer between 15 D and 25 D and the second portion of the elongate tube may be made from a material having a durometer between 40 D and 50 D.

Alternatively or additionally to any of the examples above, the first portion of the elongate tube may comprise a coil.

Alternatively or additionally to any of the examples above, the first portion of the elongate tube may comprise silicone material.

Alternatively or additionally to any of the examples above, the first portion of the elongate tube may comprise a plurality of layers, with at least one layer of the plurality of layers comprising the silicone material.

Alternatively or additionally to any of the examples above, the second portion of the elongate tube may comprise a first material and the first portion of the elongate tube may comprise an inner layer of the first material, an outer layer of a second material, and a middle layer of the silicone material positioned between the inner layer and the outer layer.

Alternatively or additionally to any of the examples above, the first portion and the second portion may have a same thickness extending from the lumen to the outer surface.

Alternatively or additionally to any of the examples above, the sheath may further include a hub, wherein the first portion of the elongate tube may have a stiffness gradient that increases in stiffness in a proximal direction toward the hub.

In another example, a repositioning sheath for replacing an introducer sheath after a blood pump is introduced into a vessel of a subject through a lumen of the introducer sheath may comprise an elongate tube having an outer diameter configured to extend through the lumen of the introducer sheath and a repositioning lumen configured to receive an elongate pump shaft extending proximally from the blood pump, wherein the elongate tube may comprise a flexible portion configured to extend through an opening in skin of the subject while a distal end of the elongate tube is in the vessel of the subject and bend to allow the elongate tube to lay along the skin of the subject while mitigating bleeding from the opening in the skin.

Alternatively or additionally to any of the examples above, the flexible portion may have a first stiffness and the elongate tube may comprise a rigid portion having a second stiffness greater than the first stiffness.

Alternatively or additionally to any of the examples above, the flexible portion may have a first stiffness and the elongate tube may comprise a first rigid portion having a second stiffness that is greater than the first stiffness and a second rigid portion having a third stiffness that is greater than the first stiffness.

Alternatively or additionally to any of the examples above, the repositioning sheath may further include a hub coupled with a proximal end of the elongate tube, wherein the hub may have a port in communication with the repositioning lumen.

Alternatively or additionally to any of the examples above, the flexible portion of the elongate tube may have a stiffness gradient that increases in stiffness in a proximal direction toward the hub.

Alternatively or additionally to any of the examples above, the flexible portion may comprise a silicone material.

Alternatively or additionally to any of the examples above, the flexible portion may comprise a coil.

In another example, a method of using a repositioning sheath may comprise advancing the repositioning sheath distally over an elongate pump shaft extending from a blood pump, through a lumen of an introducer sheath, and into a blood vessel of the subject; and removing the introducer sheath from the blood vessel of the subject, and wherein when the repositioning sheath is positioned in the blood vessel, a first portion of the elongate tube having a first stiffness may extend through an opening in skin of the subject and a second portion located distal of the first portion and having a second stiffness greater than the first stiffness may extend within the blood vessel.

Alternatively or additionally to any of the examples above, the method may further include resting a portion of the repositioning sheath proximal of the opening on the skin of the subject.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a schematic perspective view of an illustrative catheter system including a blood pump;

FIG. 2 is a schematic partial cross-section view of a blood vessel and a heart of a subject with a side view of a portion of an illustrative catheter system in the blood vessel and a blood pump positioned in the heart;

FIG. 3 is a schematic partial cross-section view of a blood vessel and a side view of a portion of an illustrative configuration of a circulatory support system;

FIG. 4 is a schematic partial cross-section view of a blood vessel and a side view of a portion of an illustrative configuration of a circulatory support system;

FIG. 5 is a schematic cross-section view of an illustrative configuration of a sheath;

FIG. 6 is a schematic cross-section view of an illustrative configuration of a sheath;

FIG. 7 is a schematic cross-section view of an illustrative configuration of a sheath;

FIG. 8 is a schematic cross-section view of an illustrative configuration of a sheath;

FIG. 9 is a schematic cross-section view of an illustrative configuration of a sheath; and

FIG. 10 is a schematic diagram of an illustrative technique for using a sheath.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “a configuration”, “some configurations”, “other configurations”, etc., indicate that the configuration described may include one or more particular features, structures, and/or characteristics. However, such recitations do not necessarily mean that all configurations include the particular features, structures, and/or characteristics. Additionally, when particular features, structures, and/or characteristics are described in connection with one configuration, it should be understood that such features, structures, and/or characteristics may also be used in connection with other configurations whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure.

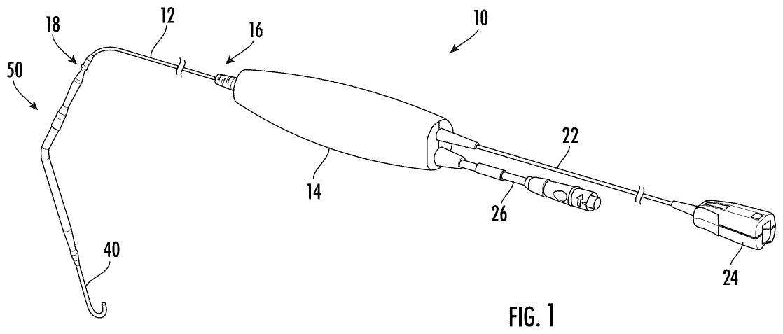

FIG. 1 illustrates a perspective view of a catheter 10 (e.g., a mechanical circulatory support system and/or other suitable catheter system) including a percutaneous blood pump 50 (e.g., a percutaneous circulatory support device, such as a percutaneous ventricular assist device (PVAD) in a left ventricular assist device (LVAD) configuration, etc.) located at a distal end region thereof. Although other suitable configurations are contemplated, the blood pump 50 may be configured to pump blood from a ventricle of a heart of a subject to vasculature of the subject.

The catheter 10 may be coupled to or may include the blood pump 50, with an elongate shaft 12 (e.g., an elongate tube with one or more elongate members extending therein) of the catheter 10 coupled with and extending proximally from the percutaneous blood pump 50 and a distal tip 40 extending distally from the blood pump 50. For instance, a proximal end 16 of the elongate shaft 12 may be coupled to a housing 14 (e.g., a housing of a handle, of a control module, etc.) and a distal end 18 of the elongate shaft 12 may be coupled to the percutaneous blood pump 50. An electrical cable 22 may extend from the housing 14 to a connector 24 at a proximal end thereof. The connector 24 may be configured to be connected to a controller (not shown) for controlling the blood pump 50, such as providing electrical power and/or control signals to the blood pump 50. The catheter 10 may also include an extension 26 connectable to the controller for sending and/or receiving signals, such as from one or more sensors during operation of the blood pump 50.

When placing the blood pump 50 in a subject (e.g., a patient and/or other suitable subject), the blood pump 50 may be inserted through an introducer sheath (e.g., a first sheath) that extends through a vascular access site in the skin of the subject and into a blood vessel. In some cases, the introducer sheath may be a large bore sheath configured to receive and facilitate the passage of the blood pump 50 and the elongate shaft 12 through a lumen thereof and into the blood vessel (e.g., a femoral artery, etc.) of the subject. Such an introducer sheath, however, may have an outer diameter that tends to block blood flow through the blood vessel (e.g., flow through the femoral artery to a leg of the subject).

To facilitate perfusion of blood to the leg and/or other portions of the subject, the introducer sheath may be swapped out for or replaced with a smaller outer diameter sheath, such as a repositioning sheath (e.g., a second sheath), after advancing the blood pump 50 into the vasculature of the subject. To facilitate removal of the introducer sheath from the vascular access site (e.g., before, during, or after placement of the repositioning sheath), the introducer sheath may be a peel-away sheath, where the introducer sheath may be configured to split along a perforated line extending along an entirety of or at least part of a length of the introducer sheath, the introducer sheath and/or the repositioning sheath may be configured such that the introducer sheath may slide over the repositioning sheath for removal, and/or the introducer sheath may be configured to be removed from the vascular access site and/or the catheter 10 in one or more other suitable manners. In some examples, the repositioning sheath may have an outer diameter smaller than a diameter of a lumen of the introducer sheath to facilitate placing the repositioning sheath in the blood vessel of the subject through the introducer sheath and via the vascular access site.

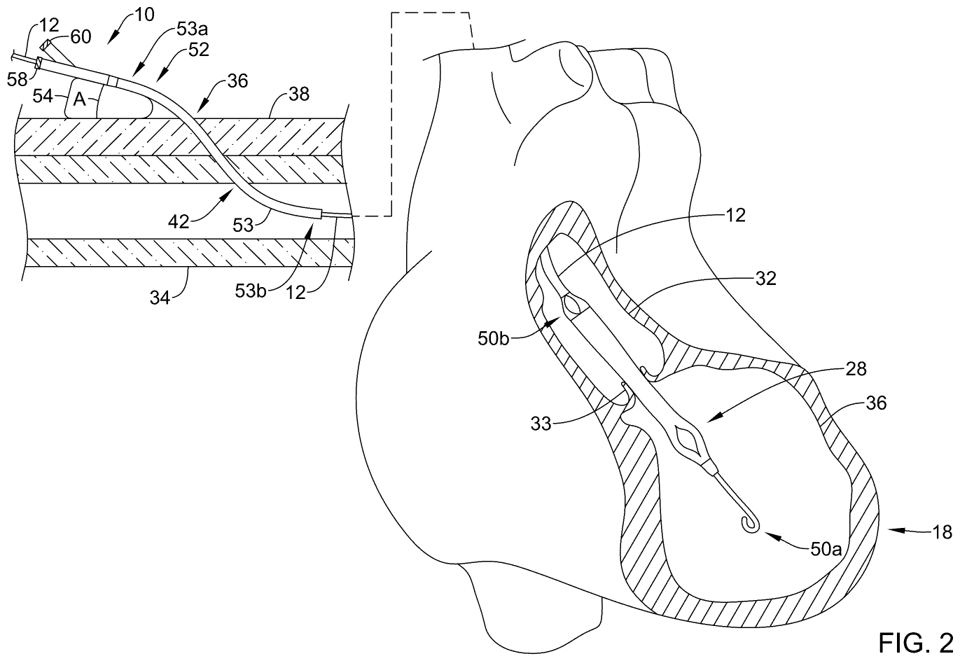

FIG. 2 depicts an illustrative positioning of the blood pump 50 in anatomy of a subject. In FIG. 2, the blood pump 50 is positioned with a distal end 50a located in a left ventricle 28 of a heart 30 and a proximal end 50b in an aorta 32, such that the blood pump 50 extends across an aortic valve 33 between the left ventricle 28 and the aorta 32. With the blood pump 50 extending from the left ventricle 28 to the aorta 32, the blood pump 50 may be configured to pump blood from the left ventricle 28 into the aorta 32 to assist or support blood flow circulation. Other suitable positions of the blood pump 50 relative to the anatomy are contemplated and include, but are not limited to, the distal end 50a of the blood pump 50 being positioned in a right ventricle of the heart 30 with a proximal end 50b being positioned in a pulmonary artery.

As depicted in FIG. 2, the elongate shaft 12 may extend from the blood pump 50 in the aorta 32 through a proximal blood vessel 34 (e.g., the femoral artery and/or other suitable blood vessel) and out of the subject. The elongate shaft 12 may extend through a repositioning sheath 52 or other suitable sheath (e.g., an introducer sheath, etc.) as the elongate shaft 12 exits the proximal blood vessel 34 and the subject to a location proximal of a hub 56 at a proximal end of an elongate tube 53 of the repositioning sheath 52, where the elongate tube 53 may extend through an opening 36 in skin 38 of the subject (e.g., a vasculature access site of the subject) and into the proximal blood vessel 34 through an opening 42 in the proximal blood vessel 34.

The repositioning sheath 52 may have any suitable configuration. In some examples, the repositioning sheath 52 may include the elongate tube 53 having a proximal end proximate the hub 56, a distal end, and one or more lumens extending between the proximal end and the distal end, along with an outer surface defining an outer diameter of the elongate tube 53. Additionally or alternatively, at least one of the one or more lumens may extend between one or more openings or ports in the elongate tube 53 between the proximal end and the distal end. In some examples, the hub 56 may be omitted.

The hub 56 may have any suitable configuration. In some examples, the hub 56 may include one or more ports. In one example, the hub 56 may include a first port 58 (e.g., a primary port configured to receive the elongate shaft 12 of the catheter 10 or other suitable device) and a second port 60 (e.g., a second port configured to receive a guidewire or other suitable device). In some examples, the first port 58 and/or the second port 60 may be in communication with one or more lumens of the elongate tube 53. Other suitable configurations are contemplated.

As the repositioning sheath 52 and the elongate shaft 12 extend proximally out of the subject, a first bending angle of the repositioning sheath 52 may be created in the repositioning sheath 52 when the repositioning sheath 52 is allowed to rest to against the skin 38 at or proximate the opening 36 through the skin 38 due to a stiffness (e.g., measured as the magnitude of force divided by displacement (e.g., newton-meters per radian, pounds per inch, etc.)) of the elongate tube 53 with the elongate shaft 12 extending therethrough. The first bending angle of the repositioning sheath 52 may cause the opening 36 in the skin 38 and/or the opening 42 in the proximal blood vessel 34 to remain open or expand (e.g., to be propped open), which may result in undesirable bleeding from the opening 36.

A spacer 54 (e.g., a wedge, gauze, etc.) may be positioned between the skin 38 of the subject or other surface supported by the skin 38 of the subject and the repositioning sheath 52 such that a second repositioning angle A between the repositioning sheath 52 and the skin 38 of the subject is greater than the first bending angle that may prop open the opening 36 through the skin 38 and/or the opening 42 of the proximal blood vessel 34. For example, the spacer 54 may be sized or otherwise configured such that the second bending angle A prevents propping open the opening 36 such that the skin may seal around the elongate tube 53. In some examples, the second bending angle A between the repositioning sheath 52 and the skin 38 of the subject that is maintained by inserting the spacer 54 therebetween may be at least thirty degrees. Further, the angle A between the repositioning sheath 52 and the skin 38 may be maintained in other suitable manners.

As repositioning sheaths 52 may be in subjects for hours or days (e.g., more than 6 hours, etc.), it can become difficult to maintain the second bending angle A using the spacer 54 between the repositioning sheath 52 and the skin 38 over time as the subject moves. As such, it may be desirable to secure the repositioning sheath 52 to the subject at or proximate the opening 36 in the skin 38 of the subject without using the spacer 54. To facilitate securing the repositioning sheath 52 to or relative to the subject at or proximate the opening 36 in the skin 38 of the subject, the repositioning sheath 52 may have a lower stiffness portion that extends through the opening 36 in the skin 38 and/or the opening 42 in the proximal blood vessel 34. In some examples, the repositioning sheath 52 may have a proximal portion with a lower stiffness (e.g., higher flexibility) than a distal portion with a higher stiffness (e.g., lower flexibility) that facilitates inserting the repositioning sheath 52 into the proximal blood vessel 34. Additionally or alternatively, the repositioning sheath 52 with the lower stiff ness portion of that extends through the opening 36 in the skin 38 and/or the opening 42 in the proximal blood vessel 34 may have higher stiffness portions of the elongate tube 53 proximal of and/or distal of the lower stiffness portion. Other suitable configurations of repositioning sheaths 52 with elongate tubes 53 having a lower stiffness portion are contemplated.

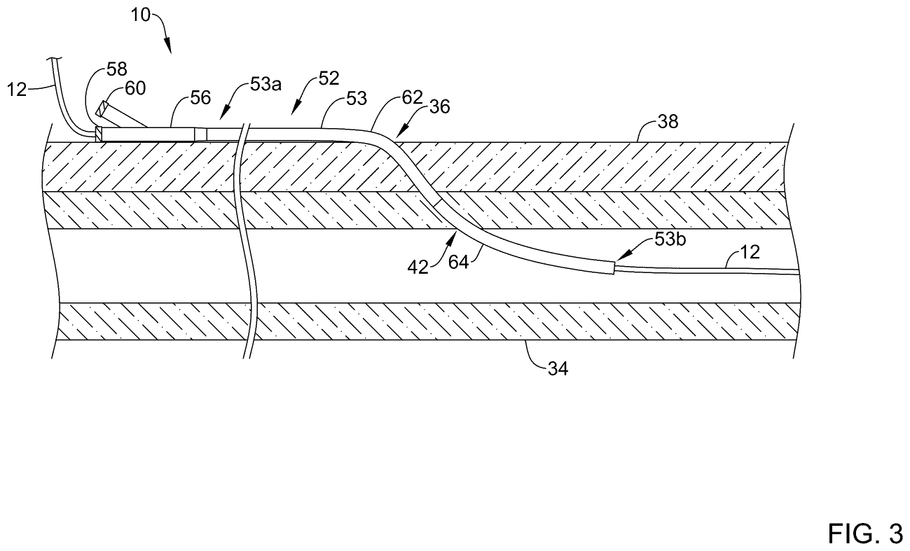

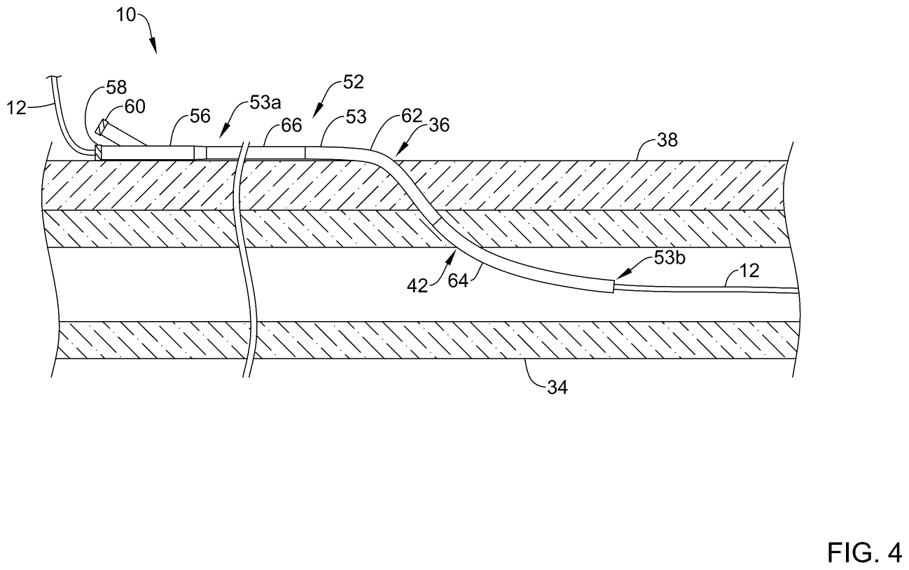

FIGS. 3 and 4 schematically depict the elongate shaft 12 of the catheter 10 extending proximally from illustrative configurations of the repositioning sheath 52 extending through the proximal blood vessel 34 and the opening 36 of the skin 38. The configurations of the repositioning sheath 52 depicted in FIGS. 3 and 4 include the elongate tube 53 with a portion having a reduced stiffness (e.g., a flexible portion) relative to at least one other portion (e.g., a rigid portion, which may be flexible enough to enter into and/or traverse a blood vessel, but less flexible or more stiff than the flexible portion) of the elongate tube 53, where the reduced stiffness portion is configured to extend through and conform to the skin 38 of the subject at the opening 36 when the repositioning sheath is positioned in the proximal blood vessel 34 of the subject. Such a configuration of the repositioning sheath 52 with the reduced stiffness portion may be configured to create a bend at or proximal of the opening 36 to allow the elongate tube 53 to lay along the skin 38 of the subject while mitigating bleeding from the opening in the skin 38. Although the repositioning sheath 52 is discussed herein as including a reduced stiffness portion relative to a more distal portion having a greater stiffness, introducer sheaths and/or other suitable sheaths may be similar configured.

The reduced stiffness portion of the elongate tube 53 of the repositioning sheath 52 may be configured to create a bend having any suitable angle for allowing the elongate tube 53 to lay along the skin 38 of the subject while mitigating bleeding from the opening in the skin 38. In some examples, the reduced stiffness portion of the elongate tube 53 may be configured to create a bend angle of ninety degrees or less, but other suitable configurations are contemplated.

The illustrative configuration of the repositioning sheath 52 depicted in FIG. 3 may include a configuration of the elongate tube 53 with a first portion 62 (e.g., a proximal portion or flexible portion) having a first stiffness and a second portion 64 (e.g., a distal portion or rigid portion) distal of the first portion 62 having a second stiffness. In some examples, the first stiffness of the first portion 62 may be less than the second stiffness of the second portion 64. The elongate tube 53 may be configured such that the first portion 62 is configured to be positioned at or extend through the opening 36 in the skin 38 and the second portion 64 may be located at and/or within the proximal blood vessel 34. Although not depicted in FIG. 3, the first portion 62 of the elongate tube may be configured to extend from exterior of the skin 38 to a location within the proximal blood vessel 34.

The illustrative configuration of the repositioning sheath 52 depicted in FIG. 4 may include a configuration of the elongate tube 53 with the first portion 62 having a first stiffness, a second portion 64 distal of the first portion 62 having a second stiffness, and a third portion 66 (e.g., a rigid portion) located proximal of the first portion 62 having a third stiffness. In some examples, the first stiffness of the first portion 62 may be less than the second stiffness of the second portion 64 and/or the third stiffness of the third portion 66 (e.g., the second stiffness and/or the third stiffness may be greater than the first stiffness). In some examples, the first stiffness of the first portion 62 may be less than the second stiffness of the second portion 64 and greater than the third stiffness of the third portion 66. In some examples, the second stiffness of the second portion 64 may be equal to or substantially equal to the third stiffness of the third portion, but other suitable configurations are contemplated.

Similar to as discussed above with respect to FIG. 3, the configuration of the elongate tube 53 depicted in FIG. 4 may be configured such that the first portion 62 is to be positioned at or extend through the opening 36 in the skin 38 when the second portion 64 is located at and/or within the proximal blood vessel 34. The third portion 66 may extend from a proximal end of the second portion to the hub 56. Although not depicted in FIG. 4, the first portion 62 of the elongate tube 53 may be configured to extend from exterior of the skin 38 to a location within the proximal blood vessel 34.

Although the first portion 62, the second portion 64, and the third portion 66 are described has having stiffnesses or stiffness levels relative to stiffnesses or stiffness levels of other portions, the stiffness levels within a portion and between portions may vary. In some examples, the elongate tube 53 may include stiffness gradients between portions of the elongate tube 53, where the stiffness of the elongate tube 53 gradually changes a between adjacent portions of the elongate tube 53 until the stiffness reaches a desired stiffness for the portion of the elongate tube 53. In some examples, the elongate tube 53 may include stiffness gradients within a portion of the elongate tube 53 such that a portion of the elongate tube 53 may have a constant stiffness over a length of the elongate tube 53 and/or a transitioning stiffness over a length of the elongate tube 53. Additionally or alternatively, when areas of gradual transitions in stiffness of the elongate tube 53 are included, the areas or portions of gradual transition in stiffness may be part of one or more of the portions of the first portion 62, the second portion 64, and the third portion 66, and/or may be an area between adjacent ones of the first portion 62, the second portion 64, and the third portion 66. In one example configuration, the first portion 62 of the elongate tube 53 may include a stiffness gradient that increases in stiffness in a proximal direction toward the hub 56. Other suitable configurations are contemplated.

Utilizing the elongate tubes 53 with a stiffer distal end or region may facilitate insertion of the repositioning sheath 52 into the proximal blood vessel 34 of the subject. For example, configurations of the elongate tube 53 including the second portion 64 with the second stiffness that is greater than the first stiffness of the first portion 62 may facilitate inserting the repositioning sheath 52 into and/or through the introducer sheath, the skin 38 of the subject, and/or the proximal blood vessel 34 of the subject by deflecting less than the first portion 62 having the first stiffness would deflect when engaging the introducer sheath, the skin 38, and/or the proximal blood vessel 34. Additionally or alternatively, the increased second stiffness of the second portion 64 of the elongate tube 53 relative to the first stiffness of the first portion 62 may have other suitable benefits.

Utilizing elongate tubes 53 with a stiffer proximal end or region may facilitate inserting devices into the repositioning sheath 52 and/or facilitate advancing or pushing the repositioning sheath 52 over the elongate shaft 12. For example, configurations of the elongate tube 53 including the third portion 66 with the third stiffness that is greater than the first stiffness of the first portion 62 may facilitate inserting devices into the repositioning sheath 52 by rigidly directing devices inserted into the hub 56 through the elongate tube 53 to the first portion 62 and the second portion 64 of the elongate tube 53. In another example, configurations of the elongate tube 53 including the third portion 66 with the third stiffness that is greater than the first stiffness of the first portion 62 may facilitate pushing the repositioning sheath 52 over the elongate shaft 12 by providing proximal rigidity as a user grasps the hub 56 and/or the third portion 66 to advance the repositioning sheath 52 in a distal direction or proximal direction.

The first portion 62 of the elongate tube 53 may have any suitable length configured to extend through the skin 38 of the subject and form a bend exterior of the vascular access site such that the repositioning sheath 52 may be secured to the skin 38 of the subject and the first portion 62 may adapt to or adjust to a shape or contour of the opening 36 through the skin 38 (e.g., to a shape or contour of the skin at the opening 36). In some examples, the length of the first portion 62 may be in a range of about 12.7 millimeters (mm) to about 6 mm. In one example, the length of the first portion 62 may be about 2 mm, but other suitable configurations are contemplated.

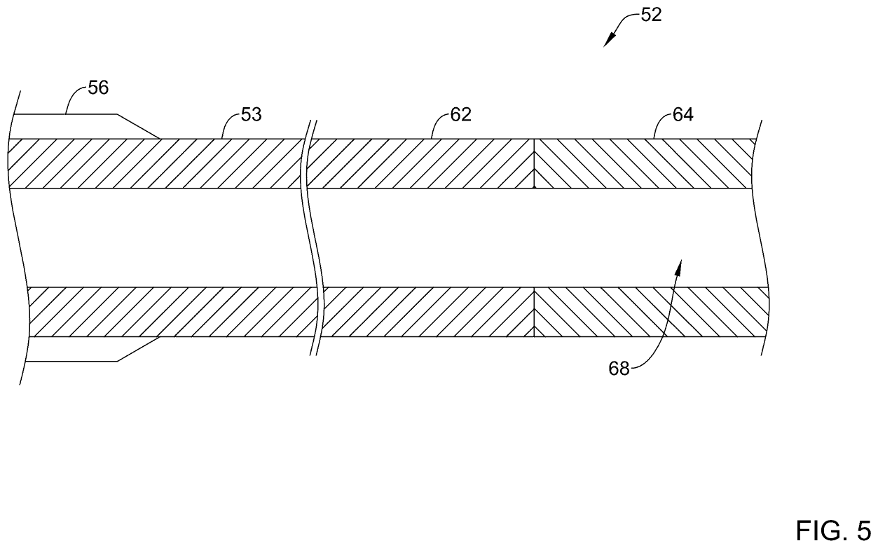

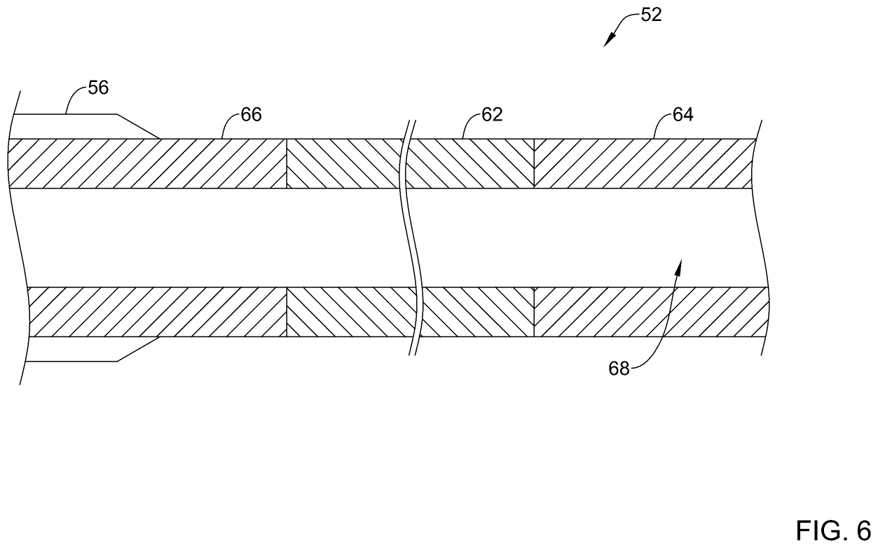

FIGS. 5 and 6 schematically depict a section of illustrative configurations of the repositioning sheath 52 including the elongate tube 52 defining one or more lumens 68 and having at least the first portion 62 of the elongate tube 53 with the first stiffness and the second portion 64 with the second stiffness. In some examples, the one or more lumens 68 may be in communication with a port of the hub 56, but this is not required. FIG. 5 schematically depicts a section of an illustrative configuration of the repositioning sheath 52 with the elongate tube 53 having the first portion 62 extending distally from the hub 56 and the second portion 64 extending distally from the first portion 62, where the second portion 64 of the elongate tube 53 may be formed from a different configuration of one or more materials than a configuration of one or more materials from which the first portion 62 may be formed. FIG. 6 schematically depicts a section of an illustrative configuration of the repositioning sheath 52 with the elongate tube 53 having the first portion 62, the second portion 64 extending distally from the first portion 62, and the third portion 66 extending distally from the hub 56 to the first portion 62, where the second portion 64 and the third portion 66 of the elongate tube 53 may be formed from a different configuration of one or more materials than a configuration of one or more materials from which the first portion 62 may be formed. In some examples, the second portion 64 and the third portion 66 may be formed from the same configuration of one or more materials (e.g., as depicted in FIG. 6) or from different configurations of one or more materials.

The first portion 62 of the elongate tube 53 may be formed from any suitable combination of one or more materials, which may be mixed together and/or form one or more layers in a radially outward direction and/or axial direction. Example suitable materials from which the first portion 62 may be formed may include, but are not limited to, polymers, metals, rubber, silicone, springs, coils, thermoplastic elastomer, polyether block amide (PEBA) (e.g., PEBAX®, VESTAMID®, etc.), thermoplastic polyurethanes (TPU) (e.g., PELLATHANE®, etc.), polytetrafluoroethylene (PTFE) (e.g., TEFLON®, etc.), a PTFE coil (e.g., a TEFLONG® coil, etc.), coils, springs, helixes, and/or other suitable materials or material configurations. In some examples, the first portion 62 may be entirely or at least partially made from a rubber and/or a silicone. Other suitable configurations are contemplated.

The first portion 62 may be made from material having any suitable hardness configured to arrive at a desired stiffness (e.g., the first stiffness, etc.) In some examples, the first portion 62 may be formed from a material having a durometer in a range of about 10 D to about 30 D, in a range of about 15 D to about 25 D, and/or other suitable durometers. In one example, the first portion 62 may be formed from a material having a durometer in a range of about 15 D to about 25 D. In one example, the first portion 62 may be formed from a material having a durometer of or about 20 D. Other suitable configurations are contemplated.

The second portion 64, the third portion 66, and/or other suitable portions of the elongate tube 53 configured to have a stiffness greater than the first stiffness of the first portion 62 may be formed from any suitable combination of one or more materials. Example suitable materials from which the first portion 62 may be formed may include, but are not limited to, polymers, metals, thermoplastic elastomers, polyether block amides (PEBA) (e.g., PEBAX®, VESTAMID®, etc.), and/or other suitable materials or material configurations. In some examples, the second portion 64 and/or the third portion 66 may be entirely or at least partially made from a PEBA. Other suitable configurations are contemplated.

The second portion 64 and/or the third portion 66 may be made from material having any suitable hardness configured to arrive at a desired stiffness (e.g., the second stiffness, the third stiffness, etc.) In some examples, the second portion 64 and/or the third portion 66 may be formed from a material having a durometer greater than about 35 D, greater than about 40 D, in a range of about 35 D to about 50 D, in a range of about 40 D to about 50 D, in a range of about 40 D to about 45 D, and/or other suitable durometers. In one example, the second portion 64 and/or the third portion 66 may be formed from a material having a durometer in a range of about 40 D to about 50 D. In one example, the second portion 64 and/or the third portion 66 may be formed from a material having a durometer of or about 40 D. In one example, the second portion 64 and/or the third portion 66 may be formed from a material having a durometer of or about 45 D. Other suitable configurations are contemplated.

In one example configuration of the elongate tube 53 as depicted in FIG. 5, the first portion 62 may be formed from a silicone material having a hardness of 20 D and the second portion 64 may be formed from a PEBA having a hardness of about 40 D or 45 D. In one example configuration of the elongate tube 53, as depicted in FIG. 6, the first portion 62 may be formed from a silicone material having a hardness of about 20 D and the second portion 64 and/or the third portion 66 may be formed from a PEBA material having a hardness of about 40 D or 45 D.

The first portion 62, the second portion 64, and the third portion 66 may have any suitable thicknesses extending from the lumen 68 to an exterior surface (e.g., may have any suitable wall thickness). As depicted in FIGS. 5 and 6, the first portion 62, the second portion 64, and the third portion 66 may have the same or substantially same thickness along a length of the elongate tube 53. Alternatively, one or more of the first portion 62, the second portion 64, and the third portion 66 may have a thickness that differs from a thickness of at least one other portion of the elongate tube 53. In one example, the first portion 62 may have a wall thickness that is less than a wall thickness of the second portion 64, the third portion 66, and/or one or more other suitable portions of the elongate tube 53, which may facilitate maintaining the elongate shaft 53 at a desired location. In another example, the first portion 62 may have a wall thickness that is greater than a wall thickness of the second portion 64, the third portion 66, and/or one or more other suitable portions of the elongate tube 53, which may facilitate plugging an opening through the skin of the patient.

The flexibility of the first portion 62, the second portion 64, and the third portion 66 of the elongate shaft 53 (e.g., relative flexibility of the portions of the elongate shaft 53), may be configured to differ from one or more other portions of the elongate shaft 53 by adjusting one or more of a material of the portion, a wall thickness of the portion, and/or a diameter of the portion of the elongate shaft 53. In one example, the first portion 62 may be configured to be less stiff (e.g., more flexible) than the second portion 64 and/or the third portion 66 by configuring each portion 62, 64, 66 with the same wall thickness and same outer diameter, while forming the first portion 62 with a lower durometer material (e.g., a lower durometer single material or a combination of materials that result in a lower durometer) than a durometer of the material used to form the second portion 64 and/or the third portion 66. In another example, the first portion 62 may be configured to be less stiff (e.g., more flexible) than the second portion 64 and/or the third portion 66 by configuring the first portion 62 from a first material with a lower durometer than the material of the second portion 64 and/or the material of the third portion 66, using a larger outer diameter than an outer diameter of the second portion 64 and/or the third portion 66, and using a smaller wall thickness than the wall thickness of the second portion 64 and/or the third portion 66. In another example, the first portion 62 may be configured to be less stiff (e.g., more flexible) than the second portion 64 and/or the third portion 66 by configuring the first portion 62, the second portion 64, and the third portion 66 from a same or similar material, using a smaller outer diameter for the first portion 62 than an outer diameter of the second portion 64 and/or the third portion 66, and using a smaller wall thickness of the first portion 62 than the wall thickness of the second portion 64 and/or the third portion 66. Other suitable configurations of the portions 62, 64, 66 are contemplated for adjusting the relative stiffness or flexibility of portions of the elongate shaft 53.

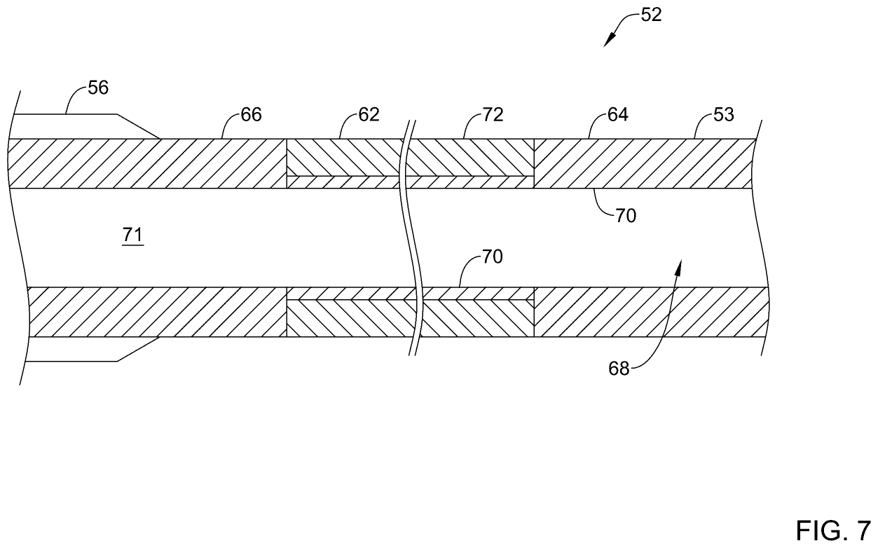

FIGS. 7-9 depict illustrative configurations of the repositioning sheath 52 with the elongate tube 53 having the first portion 62, the second portion 64, and the third portion 66. In some examples, the first portion 62, however, may extend proximally to at least the hub 56 and the third portion 66 may be omitted, as desired. Each of the first portion 62, the second portion 64, and the third portion 66, when included, may be formed of one or more layers of material. The vertical lines separating the first portion 62 from the second portion 64 and the third portion 66 are depicted for clarity and may or may not be visible in a cross-section of the repositioning sheath 52.

As depicted in FIG. 7, the second portion 64 and the third portion 66 of the elongate tube 53 may be formed from at least a same first material 70, but other suitable configurations are contemplated. The first portion 62 of the elongate tube 53 may be formed from a combination of materials (e.g., a plurality of materials in one or more layers or mixes). In some examples, the first portion 62 may be formed from the first material 70 and a second material 72. In one example configuration of the elongate tube 53, the first material 70 may be or may include a PEBA material having a hardness of or about 40 D or 45 D and the second material 72 may be or may include a silicon material having a hardness of or about 20 D. Other suitable configurations of the elongate tube 53 are contemplated.

As depicted in FIG. 7, the materials of the first portion 62 may form a plurality of layers with the first material 70 forming an inner layer (e.g., with an inner surface 71) entirely or at least partially defining the lumen 68 of the elongate tube 53 along the first portion 62, the second portion 64, and the third portion 66 and the second material 72 forming an outer layer entirely or at least partially defining an outer surface of the first portion 62 of the elongate tube 53. In some examples, the inner layer of the first material 70 at the first portion 62 of the elongate tube 53 may be a thin layer relative to the outer layer of the second material 72 and may facilitate maintaining a lubricious inner diameter to facilitate movement of the elongate shaft 12 of the catheter and/or other medical devices within the repositioning sheath 52. When making the elongate tube 53, the second material 72 may be added on top of the thin strip of first material 70 at the first portion 62 of the elongate tube 53 and the second material 72 may extend outward to form an outer surface of the elongate tube 53 that is in-line with the outer surface of the elongate tube 53 at the second portion 64 and the third portion 66, but other suitable techniques for forming the elongate tube 53 are contemplated.

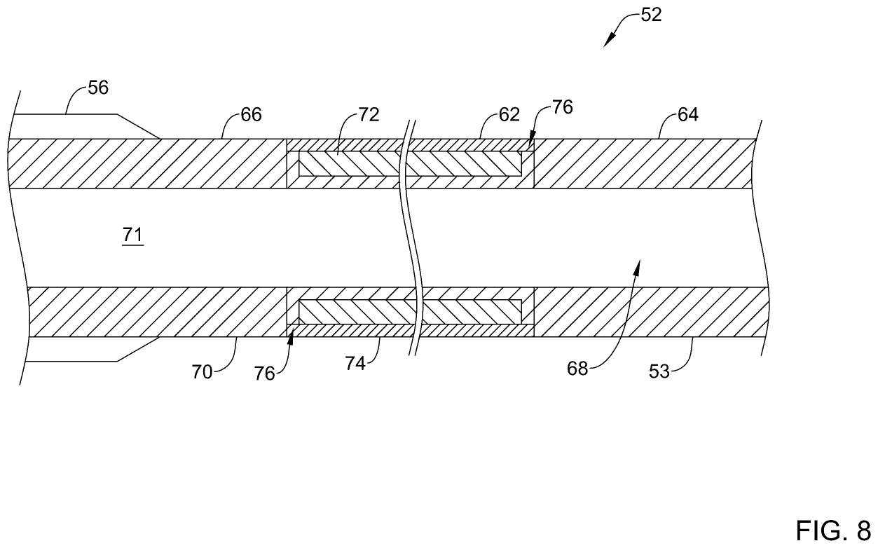

As depicted in FIG. 8, the second portion 64 and the third portion 66 of the elongate tube 53 may be formed form a same first material 70, but other suitable configurations are contemplated. The first portion 62 of the elongate tube 53 may be formed from a combination of materials. In some examples, the first portion 62 may be formed from the first material 70, the second material 72, and a third material 74. In one example configuration of the elongate tube 53, the first material 70 may be or may include a PEBA material having a hardness of or about 40 D or 45 D, the second material 72 may be or may include a silicone material having a hardness of or about 20 D, and the third material 74 may be a TPU. Other suitable configurations of the elongate tube 53 are contemplated.

As depicted in FIG. 8 and similar to as discussed with respect to FIG. 7, the materials of the first portion 62 may form a plurality of layers with the first material 70 forming an inner layer (e.g., with the inner surface 71) entirely or at least partially defining the lumen 68 of the elongate tube 53 along the first portion 62, the second portion 64, and the third portion 66. In some examples, the second material 72 may form a middle layer of the first portion 62 and the third material 74 may form an outer layer entirely or at least partially defining an outer surface of the first portion 62 of the elongate tube 53. In some examples, the inner layer of the first material 70 and the outer layer of the third material 74 at the first portion 62 of the elongate tube 53 may be thin layers of material relative to the middle layer of the second material 72, which may form a majority of the thickness of the first portion 62 of the elongate tube 53. The inner layer of the first material 70 may facilitate maintaining a lubricious inner diameter, the middle layer of the second material 72 may facilitate flexibility of the elongate tube 53, and the outer layer of the third material 74 may be configured to ensure smooth transitions between the materials forming the outer surfaces of the first portion 62, second portion 64, and the third portion 66 of the elongate tube 53. When making the elongate tube 53, the first material 70 may be formed to have a step 76 at a proximal end and/or a distal end of the first portion 62 such that the second material 72 may be applied on the first material 70 up to the step 76 and the third material 74 may extend over the second material 72 and the step 76 of the first material 70, but other suitable techniques for forming the elongate tube 53 are contemplated.

In some examples, an outer layer of TPU may be applied to the first portions 62 of other configurations of the elongate tube 53. For example, the elongate tube 53 may have an inner layer and/or one or more middle layers formed of silicone and/or one or more other suitable materials and a layer of TPU may be applied to the inner and/or middle layers to form a smooth outer transition between the first portion 62 and one or more other portions of the elongate tube 53.

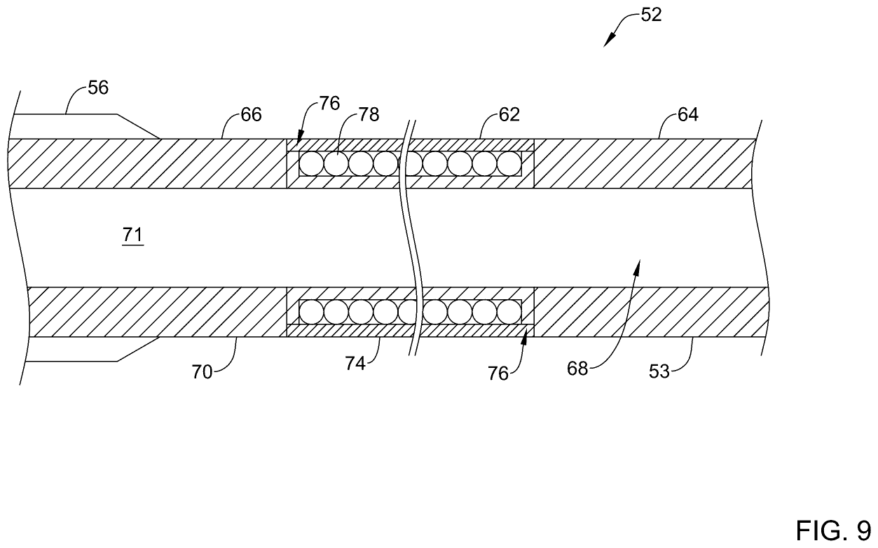

As depicted in FIG. 9, the second portion 64 and the third portion 66 of the elongate tube 53 may be formed form a same first material 70, but other suitable configurations are contemplated. The first portion 62 of the elongate tube 53 may be formed from a combination of materials. In some examples, the first portion 62 may be formed from the first material 70, a flexible helix or coil 78, and the third material 74. In one example configuration of the elongate tube 53, the first material 70 may be or may include a PEBA material having a hardness of or about 40 D or 45 D, the helix or coil 78 may be formed from a PTFE material, and the third material 74 may be a TPU. Other suitable configurations of the elongate tube 53 are contemplated. Although not depicted and not required, the helix or coil 70 may be surrounded with a silicone material, rubber material, and/or other suitable flexible material.

As depicted in FIG. 9 and similar to as discussed with respect to FIGS. 7 and 8, the materials of the first portion 62 may form a plurality of layers with the first material 70 forming an inner layer (e.g., with the inner surface 71) entirely or at least partially defining the lumen 68 of the elongate tube 53 along the first portion 62, the second portion 64, and the third portion 66. In some examples, the helix or coil 78 may form a middle layer of the first portion 62 and the third material 74 may form an outer layer entirely or at least partially defining an outer surface of the first portion 62 of the elongate tube 53. In some examples, the inner layer of the first material 70 and the outer layer of the third material 74 at the first portion 62 of the elongate tube 53 may be thin layers of material relative to the middle layer of the helix or coil 78, which may form a majority of the thickness of the first portion 62 of the elongate tube 53. The inner layer of the first material 70 may facilitate maintaining a lubricious inner diameter or surface, the middle layer of the helix or coil 78 may facilitate flexibility and pushability of the elongate tube 53, and the outer layer of the third material 74 may be configured to ensure smooth transitions between materials forming the outer surfaces of the first portion 62, second portion 64, and the third portion 66 of the elongate tube 53. When making the elongate tube 53, the first material 70 may be formed to have a step 76 at a proximal end and a distal end of the first portion 62 such that the helix or coil 78 may be applied on the first material 70 up to the step 76 and the third material 74 may extend over the helix or coil 78 and the step 76 of the first material 70, but other suitable techniques for forming the elongate tube 53 are contemplated.

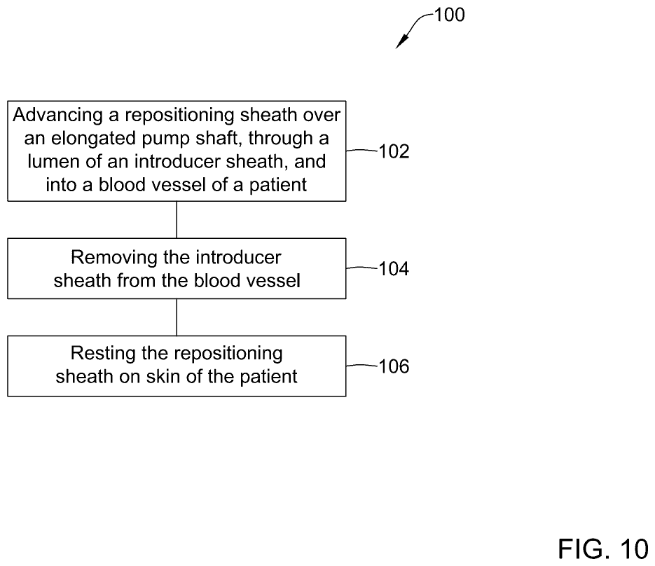

FIG. 10 schematically depicts an illustrative technique 100 for using a repositioning sheath with a circulation support system including a blood pump configured to be placed in a heart of a subject (e.g., a patient and/or other suitable subject) and an elongate shaft (e.g., an elongate pump shaft) extending proximal from the blood pump through vasculature of the subject and out of a vascular access site. In operation, the blood pump and the elongate shaft may be inserted into the vasculature of the subject through an introducer sheath inserted in a vascular access site of the subject and extending into a blood vessel of the vasculature. Once the blood pump has been inserted into the vasculature and/or positioned in or at the heart of the subject, the introducer sheath may be replaced with a repositioning sheath extending into the blood vessel and through the vasculature access site, where the repositioning sheath may have an outer diameter that is smaller than an outer diameter of the introducer sheath.

The technique 100 may include advancing 102 a repositioning sheath over or along the elongate shaft extending proximally from the blood pump, through a lumen of the introducer sheath, and into a blood vessel of the subject. In some example configurations, the introducer sheath may extend through the vasculature access site and into the blood vessel when the repositioning sheath is inserted into the lumen of the introducer sheath to facilitate placing the repositioning sheath within the blood vessel. Alternatively, in some examples, the repositioning sheath may be advanced over or along the elongate shaft and into the blood vessel of the subject without being passed through the introducer sheath.

The repositioning sheath may be any suitable repositioning sheath discussed herein or otherwise. In some examples, the repositioning sheath may have a flexible portion configured to extend through the vasculature access site such that the repositioning sheath conforms to the vasculature access site and does not prop open the vasculature access site. Although other suitable configurations are contemplated, the repositioning sheath may have one or more portions that are formed to be more stiff than the flexible portion, where the more stiff portions may be distal of and/or proximal of the flexible portion.

The technique 100 may include removing 104 the introducer sheath from the blood vessel in which it was positioned for the purpose of advancing the circulatory support system into the vasculature of the subject. In some examples, the introducer sheath may be withdrawn or otherwise removed from the blood vessel and the vasculature access site after, prior to, and/or during insertion of the repositioning sheath in the blood vessel.

The introducer sheath may be removed from the vasculature access site and blood vessel in any suitable manner. In one example, the introducer sheath may be removed from the vasculature access site and the blood vessel by withdrawing the introducer sheath over the elongate shaft of the circulatory support system and then peeling the introducer sheath apart to fully remove the introducer sheath from the circulatory support system. In another example, the introducer sheath may be withdrawn from the vasculature access site and the blood vessel by sliding the introducer sheath over the elongate shaft and maintaining introducer sheath over the elongate shaft while the circulatory support system is within the subject. The introducer sheath may be removed from the vasculature access site and the blood vessel in one or more other suitable manners, as desired.

The technique 100 may include resting 106 the repositioning sheath on skin of the subject while the repositioning sheath is positioned in the blood vessel and extending proximally from the vascular access site. Due to the flexible portion of the repositioning sheath extending through the vasculature access site (e.g., which may include an opening in and/or through the skin of the subject), the repositioning sheath may be configured to sufficiently bend without propping open the vasculature access site such that a portion of the repositioning sheath proximal of the vasculature access site may be supported by and/or secured to (e.g., directly supported by and/or directly secured to) the skin of the subject while the blood pump of the circulatory support system is positioned in the subject.

Once the repositioning sheath is positioned within the blood vessel, one or more devices may be inserted into a lumen of the repositioning sheath and/or adjusted relative to the repositioning sheath. In one example, the blood pump at the heart of the subject may be adjusted or replaced by distally and/or proximally advancing the elongate shaft extending from the blood pump and through the repositioning sheath. In one example, a guidewire may be inserted through a lumen of the repositioning sheath as desired to maintain a wire through the vascular access site and the blood vessel and/or to block blood from entering the lumen. The repositioning sheath may be utilized in one or more other suitable manners.

Using the technique 100 and a repositioning sheath with a flexible portion configured to extend through the vasculature access site, may allow for omitting a spacer or support for supporting the repositioning sheath and/or the circulatory support system proximal of the vasculature access site without propping open the vasculature access site. Omitting the spacer or support may facilitate stabilizing the circulatory support system proximal of the vasculature access site, which may mitigate unintentional movement of the elongate shaft that could cause undesired movement or positioning of the blood pump in the heart of the subject. As circulatory support systems may be positioned within the subject for an extended period of time (e.g., at least six hours or one or more days), mitigating unintentional movement of the elongate shaft may be desirable.

The schematic methods and techniques discussed herein may be used together, unless expressly indicated otherwise. Further, the order of implementation of the steps discussed herein may be in other suitable orders, unless expressly indicated otherwise, and/or the methods and techniques may include one or more intervening steps discussed herein or otherwise.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A sheath, comprising:
an elongate tube, the elongate tube having a proximal end, a distal end, a lumen extending between the proximal end and the distal end, and an outer surface defining an outer diameter, and
wherein the outer diameter is configured to pass through an introducer lumen of an introducer sheath and the elongate tube has a first portion with a first stiffness and a second portion located distal of the first portion with a second stiffness that is greater than the first stiffness.

2. The sheath of claim 1, wherein the elongate tube has a third portion located proximal of the first portion with a third stiffness that is greater than the first stiffness.

3. The sheath of claim 1, wherein the first portion is configured to extend through an opening in a skin of a subject when the second portion extends into a blood vessel of the subject.

4. The sheath of claim 3, wherein the first stiffness of the first portion of the elongate tube is configured to adapt to a contour of skin of the subject at the opening in the skin.

5. The sheath of claim 1, wherein the first portion of the elongate tube is made from a material having a durometer between 15 D and 25 D and the second portion of the elongate tube is made from a material having a durometer between 40 D and 50 D.

6. The sheath of claim 1, wherein the first portion of the elongate tube comprises a coil.

7. The sheath of claim 1, wherein the first portion of the elongate tube comprises silicone material.

8. The sheath of claim 7, wherein the first portion of the elongate tube comprises a plurality of layers, with at least one layer of the plurality of layers comprising the silicone material.

9. The sheath of claim 7, wherein the second portion of the elongate tube comprises a first material and the first portion of the elongate tube comprises an inner layer of the first material, an outer layer of a second material, and a middle layer of the silicone material positioned between the inner layer and the outer layer.

10. The sheath of claim 1, wherein the first portion and the second portion have a same thickness extending from the lumen to the outer surface.

11. The sheath of claim 1, further comprising:
a hub, and
wherein the first portion of the elongate tube has a stiffness gradient that increases in stiffness in a proximal direction toward the hub.

12. A repositioning sheath for replacing an introducer sheath after a blood pump is introduced into a vessel of a subject through a lumen of the introducer sheath, the repositioning sheath comprising:
an elongate tube having an outer diameter configured to extend through the lumen of the introducer sheath and a repositioning lumen configured to receive an elongate pump shaft extending proximally from the blood pump, and
wherein the elongate tube comprises a flexible portion configured to extend through an opening in skin of the subject while a distal end of the elongate tube is in the vessel of the subject and bend to allow the elongate tube to lay along the skin of the subject while mitigating bleeding from the opening in the skin.

13. The repositioning sheath of claim 12, wherein the flexible portion has a first stiffness and the elongate tube comprises a rigid portion having a second stiffness greater than the first stiffness.

14. The repositioning sheath of claim 12, wherein the flexible portion has a first stiffness and the elongate tube comprises a first rigid portion having a second stiffness that is greater than the first stiffness and a second rigid portion having a third stiffness that is greater than the first stiffness.

15. The repositioning sheath of claim 12, further comprising:
a hub coupled with a proximal end of the elongate tube, and
wherein the hub has a port in communication with the repositioning lumen.

16. The repositioning sheath of claim 15, wherein the flexible portion of the elongate tube has a stiffness gradient that increases in stiffness in a proximal direction toward the hub.

17. The repositioning sheath of claim 12, wherein the flexible portion comprises a silicone material.

18. The repositioning sheath of claim 12, wherein the flexible portion comprises a coil.

19. A method of using a repositioning sheath, the method comprising:
advancing the repositioning sheath distally over an elongate pump shaft extending from a blood pump, through a lumen of an introducer sheath, and into a blood vessel of a subject; and
removing the introducer sheath from the blood vessel of the subject, and
wherein when the repositioning sheath is positioned in the blood vessel, a first portion of an elongate tube of the repositioning sheath having a first stiffness extends through an opening in skin of the subject and a second portion of the elongate tube located distal of the first portion and having a second stiffness greater than the first stiffness extends within the blood vessel.

20. The method of claim 19, further comprising:
resting a portion of the repositioning sheath proximal of the opening on the skin of the subject.
